(19) United States
(12) Patent Application Publication
(10) Pub. No.: US 2009/0326418 A1
(43) Pub. Date: Dec. 31, 2009

(54) MICROPHONE MATRIX FOR RECORDING BODY SOUNDS

(75) Inventors: Gregory Gelman, Rehovot (IL); Nissim Varon, Kfar-saba (IL); Igal Kushnir, Pardes Hana (IL); Edi Marochnik, Netanya (IL)

Correspondence Address: THE NATH LAW GROUP, 112 South West Street, Alexandria, VA, 22314, US

(73) Assignee: DEEPBREEZE LTD., Or Akiva (IL)

(21) Appl. No.: 12/310,030

(22) PCT Filed: Aug. 7, 2007
(86) PCT No.: PCT/IL2007/000985

§ 371 Date: Jun. 30, 2009

Related U.S. Application Data
(60) Provisional application No. 60/835,892, filed on Aug. 7, 2006.

(51) Int. Cl. A61B 7/00

(52) U.S. Cl. 600/586

(57) ABSTRACT

The invention provides a microphone matrix for recording body sounds, such as respiratory tract sounds. The matrix has a frame having one or more recesses or openings on one of its surfaces. A microphone assembly is disposed in each recess or opening in the frame. When a body portion is applied to the matrix, each microphone assembly moves from an extended, spring biased position in which at least a portion of the microphone assembly protrudes beyond the surface of the frame towards a retracted position in which the microphone assembly is deeper inside the recess.

### FIELD OF THE INVENTION

This invention relates to medical devices and more particularly to such devices for applying a microphone to a body surface.

### BACKGROUND OF THE INVENTION

Body sounds are routinely used by physicians in the diagnosis of various disorders. A physician may place a stethoscope on a person's chest or back and monitor the person's breathing or heart sounds in order to detect adventitious (i.e. abnormal or unexpected) body sounds. The identification and classification of these adventitious sounds often provide important information about physiological abnormalities.

It is also known to affix a microphone to the body in order to record body sounds. The recorded sound signals may be amplified and filtered before being listened to by the physician. The recorded signals may also be analyzed by signal processing techniques.

It is also known to affix a plurality of microphones over a body surface in order to obtain a plurality of sound signals simultaneously from the body surface. Applicant's U.S. Pat. No. 6,887,208 discloses a system in which a plurality of microphones are affixed to a person's back or chest for recording respiratory tract sounds. This patent teaches embedding the microphones in a matrix that may be in the form of a vest or garment securely worn by the person during signal acquisition. Different sized or shaped matrixes may be used for differently sized individuals, for different sexes, ages, etc.

U.S. Pat. No. 6,394,967 discloses a system in which a plurality of microphones are affixed to a person's back or chest for recording respiratory tract sound. This patent teaches affixing the microphones to the body surface using tape or straps to prevent dislocation or movement during the data acquisition process.

Kompis et al. (Chest 120:4, 2001, 1309-1321) discloses affixing eight or sixteen microphones to an individual's chest for obtaining respiratory tract signals, but the method of attachment is not disclosed.

U.S. Pat. No. 4,777,961 discloses affixing a microphone to a body surface by means of suction. A microphone embedded in the wall of a dome shaped stethoscope head is formed from an elastic flexible material. The stethoscope head is squeezed by fingertip pressure as it is applied to the body surface. When the fingertip pressure is removed, the head expands slightly so as to create a partial vacuum in its interior so as to keep the head affixed to the body surface. The membrane of the microphone, however, is not pressed against the body surface.

U.S. Pat. No. 4,736,749 discloses a holder for a signal pick-up device, such as a microphone or electrode that is fixed to the body surface by vacuum. The holder has a chamber that is evacuated by an external source of negative pressure. When the chamber is evacuated, the holder is held firmly to the skin and the signal-pick up device is pressed to the skin inside the chamber.

U.S. Pat. No. 6,790,183 discloses embedding a plurality of microphones within a single structure referred to as a “cassette”. The cassette includes a core preferably made from foam, for supporting the microphones. Recesses formed in the foam core receive the microphones so that the microphones are flush with the upper surface of the core. The cassette is positioned on a hospital bed between the mattress and a patient and the patient lies on the cassette with the microphones positioned at a location of interest to for the recording of body sounds.

U.S. Pat. No. 7,011,087 discloses a mat that is placed beneath a lying patient. The mat contains measuring means to measure airway vibrations of the patient.

### SUMMARY OF THE INVENTION

In its first aspect, the present invention provides a microphone matrix for recording body sounds. The microphone matrix of the invention comprises a housing having a surface with a plurality of recesses. A microphone assembly is suspended in each recess by means of one or more elastic elements. The elastic elements position the microphone assembly in a position in which at least a portion of the microphone assembly protrudes beyond the surface of the housing. When a force is applied to the microphone assembly towards the housing, the microphone assembly moves against the elasticity of the elastic elements towards the interior of the housing. Thus, when a body surface, such as a person's back is applied to the matrix, the microphone assemblies are pressed onto the body surface to create good acoustic coupling between the body surface and the microphone assembly.

The housing and the elastic elements are preferably formed from an acoustically dampening material. A spacer in each microphone assembly is preferably formed from an acoustically conducting material. Thus vibrations received in the spacer are conducted to the microphone while vibrations in the housing or the projection are essentially not conducted to the microphone. Cross-talk between the microphones is also reduced.

In one embodiment of the invention, the microphone matrix comprises a plurality of microphone units joined together by a flexible frame. In another embodiment, the microphone matrix has a housing contoured to receive a body portion from which body sounds are to be recorded. For example, the surface may be contoured so as to receive the back and neck region of the body when respiratory sounds are to be detected. A microphone is suspended in each of a plurality of recesses in the surface by means of two or more elastic elements, as explained above.

In its second aspect, the invention provides a system for recording and/or analyzing body sound signals. The system of the invention includes one or more microphone matrices of the invention. Each microphone in the matrix produces an analog voltage signal indicative of pressure waves arriving at that microphone. In use, the matrix is placed on a surface such as an examination table or hospital bed. Since the microphones in the microphone matrix are elastically biased to protrude beyond the upper surface of the matrix housing, with a body surface applied to the surface of the housing, at least some of the weight of the individual is transferred to the microphones. In this way, the microphones are firmly applied to the body surface without the need of an attachment system, such as straps, tape, or vacuum. Respiratory tract sounds, or other body sounds at the body surface are detected by the microphones. The system of the invention also includes signal processing circuiting for processing signals obtained by the microphones, as required in any application. For example, the electronic circuitry for processing acoustic signals obtained from two or more microphones, as disclosed in U.S. Pat. No. 6,394,967 to Murphy, Kompis et al. supra, and in Applicant's U.S. Pat. No. 6,887,208 may be used in the system of the invention.

In its third aspect, the invention provides a method for recording body sounds. In accordance with this aspect of the invention, a body surface is applied to a microphone matrix of the invention and sounds detected by the microphones are recorded and/or analyzed.

This, in its first aspect, the invention provides a microphone matrix for recording body sounds, comprising:
(a) a frame having one or more recesses or openings on a surface of the housing or frame; and
(b) one or more microphone assemblies, each microphone assembly comprising a microphone, and each microphone assembly being disposed in a recess or opening in the frame, and wherein each microphone assembly is movable from an extended, spring biased position in which at least a portion of the microphone assembly protrudes beyond the surface of the frame to a retracted position in which the microphone assembly is deeper inside the recess.

In its second aspect, the invention provides a method for recording body sounds comprising:
(a) providing a microphone matrix for recording body sounds, the matrix comprising:
   (i) a frame having one or more recesses or openings on a surface of the housing or frame; and
   (ii) one or more microphone assemblies, each microphone assembly comprising a microphone, and each microphone assembly being disposed in a recess or opening in the frame, and wherein each microphone assembly is movable from an extended, spring biased position in which at least a portion of the microphone assembly protrudes beyond the surface of the frame to a retracted position in which the microphone assembly is deeper inside the recess.
(b) placing the microphone matrix on a surface; and
(c) applying a body surface of an individual to the surface of the frame.

In its third aspect, the invention provides a system for analyzing body sounds comprising:
(a) a microphone matrix for recording body sounds, the matrix comprising:
   (i) a frame having one or more recesses or openings on a surface of the housing or frame; and
   (ii) one or more microphone assemblies, each microphone assembly comprising a microphone, and each microphone assembly being disposed in a recess or opening in the frame, and wherein each microphone assembly is movable from an extended, spring biased position in which at least a portion of the microphone assembly protrudes beyond the surface of the frame to a retracted position in which the microphone assembly is deeper inside the recess; and
(b) electronic circuitry for analyzing signals obtained by the one or more microphones.

The invention also provides a microphone assembly for use in the microphone matrix of the invention.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a perspective view of a microphone unit for use in a microphone matrix of the invention;

FIG. 2 shows the projection of the microphone unit of FIG. 1;

FIG. 3 shows a cross-section of the microphone unit of FIG. 1 in the absence of a force applied to the microphone assembly;

FIG. 4 shows a cross-section of the microphone unit of FIG. 1 in the presence of a force applied to the microphone assembly;

FIG. 5 shows a microphone matrix in accordance with one embodiment of the invention comprising the microphone unit of FIG. 1;

FIG. 6 shows a microphone matrix in accordance with another embodiment of the invention having a contoured housing;

FIG. 7 shows a section of the microphone matrix of FIG. 6;

FIG. 8 shows the microphone matrix of FIGS. 6 and 7 in the presence of a force applied to the microphone assemblies;

FIG. 9 shows a system for recording and analyzing body sounds in accordance with one embodiment of the invention; and

FIG. 10 shows exemplary electronic circuitry for use in the system of FIG. 9.

### DETAILED DESCRIPTION OF EXEMPLARY EMBODIMENTS

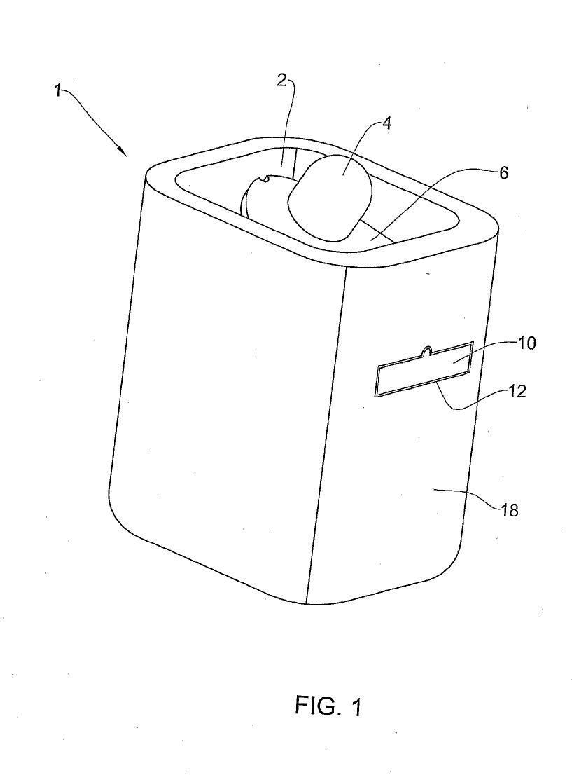

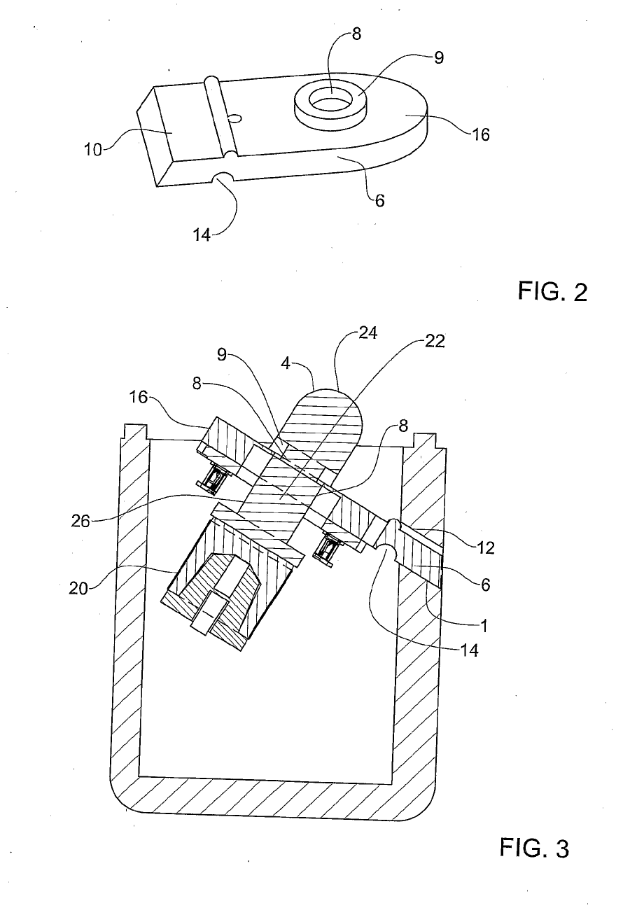

FIG. 1 shows a microphone unit 1 for use in a microphone matrix for recording body sounds in accordance with one embodiment of the invention. The microphone unit 1 has a housing having the general shape of a rectangular parallelepiped. The housing 1 has an opening 2 on an upper side. A microphone assembly 4 is supported by a projection 6 shown alone in FIG. 2. The projection 6 has an aperture 8 surrounded by a collar 9 configured to receive the microphone assembly 4. The projection 6 has a tab portion 10 configured to be inserted into a slot 12 formed into the wall of the housing 1, as shown in FIG. 1. A groove 14 is formed in the bottom surface of the projection 6 that separates the tab portion 10 from a tongue portion 16. The groove 14 creates a resiliently flexible hinge at the boundary between the tab portion 10 and the tongue portion 16.

FIG. 3 shows a cross-sectional view of the microphone unit 1 through the microphone assembly 4. The projection 6 is inserted into the slot 12 and is bonded in place. The slot 12 is formed in a wall 18 of the housing at an oblique angle so that the projection 6 extends obliquely from the wall 18. The microphone assembly 4 includes a microphone 20 and a spacer 22. The spacer 22 has a narrow neck portion 26 configured to be immobilized in the aperture 8 in a snap-fit, and a dome portion 24 having a hemispherical surface.

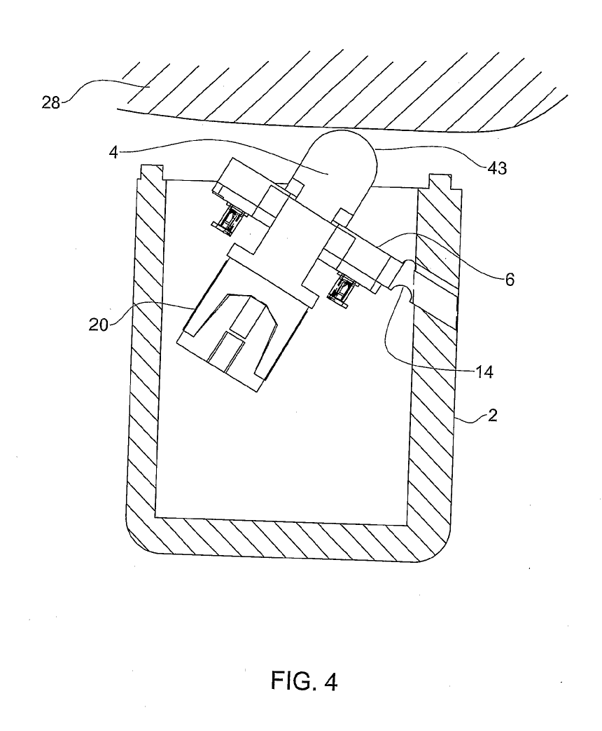

The hinge in the projection 6 formed by the groove 14 and is biased in a straight position, as shown in FIG. 3, when an external force is not applied the dome portion 24. In this configuration, the dome portion 24 protrudes beyond the walls of the housing 2. When a downward force is applied to the dome portion 24, the projection 6 bends at the hinge 14, with the tongue portion 16 moving downwards towards the interior of the housing 2. For example, as shown in FIG. 4, when a body part 28, such as a person's back is applied to the dome portion 24, the tongue portion 16 is deflected downwards. Due to the resiliently flexible character to the hinge 14, the dome portion 24 is pressed onto the surface of the body part 28 to create good acoustic coupling between the body part 28 and the dome portion 24.

The housing 2 and the projection 6 are formed from an acoustically dampening material. The spacer 22 is formed from an acoustically conducting material. Thus vibrations received in the dome portion 24 of the spacer 22 are conducted to the microphone 20. Vibrations received in the housing 2 or the projection 6 are essentially not conducted to the microphone 20. Thus, vibrations originating in the body part 28 are received in the dome portion 24 and conducted to the microphone 20.

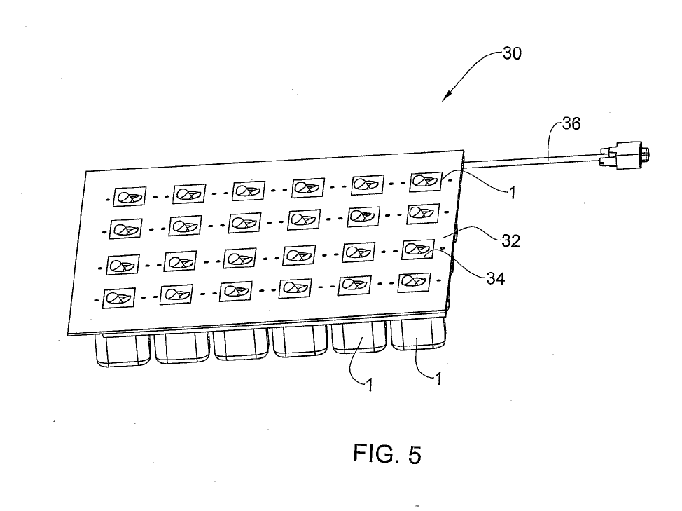

FIG. 5 shows a microphone matrix 30 comprising a plurality of the microphone units 1. The matrix 30 has a frame 32 formed from a flexible acoustically dampening material in which a plurality of apertures 34 are formed. Each aperture 34 is configured to receive a microphone unit 1 that is grasped in the aperture by a snap fit. Due to the flexibility of the frame 32, the matrix 30 is flexible and can conform to the shape of a surface applied to it. For example, when the matrix is placed on a mattress and person's back is applied to the matrix, the matrix will conform to the shape of the person's back. The matrix 30 includes a cable 36 of electrical wire leads from each microphone. In another embodiment (not shown), the microphone leads are in the form of a printed circuit on the surface of the support 2. In yet another embodiment (not shown), the microphones 4 are wireless, in which case each microphone includes a transmitter for transmitting signals to the electronic circuitry.

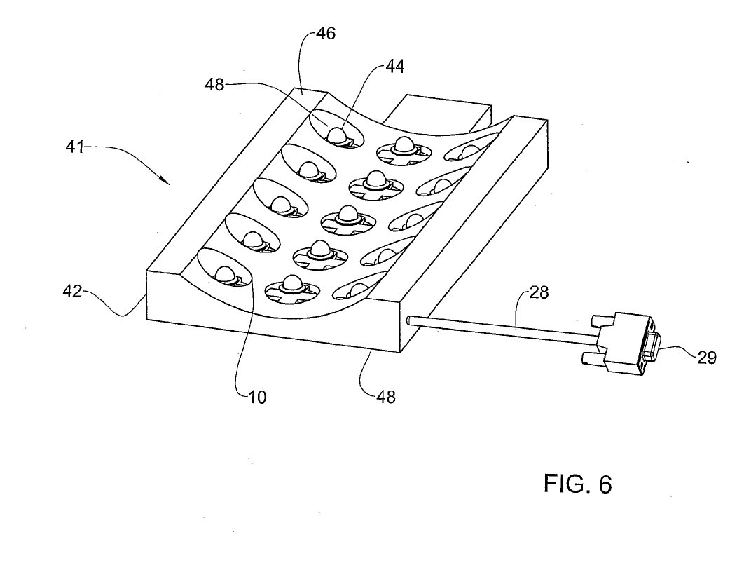

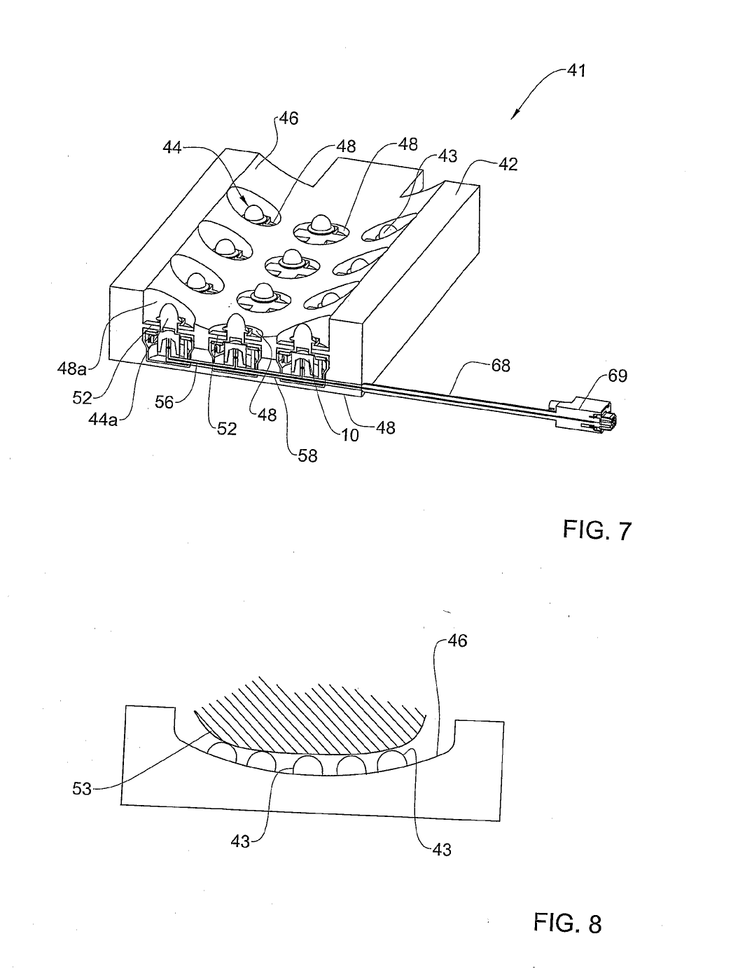

FIGS. 6 and 7 show a microphone matrix 41 for recording body sounds in accordance with another embodiment of the invention. The matrix 41 is shown in a perspective view in FIG. 6 and in a sectional view in FIG. 7. The matrix 41 includes a frame 42 in which one or more microphones 44 are embedded. The frame 42 has an upper surface 46 that is contoured to receive a body portion from which body sounds are to be recorded. For example, as shown in FIG. 6, the upper surface 46 may be contoured so as to receive the back and neck region of the body when respiratory tract sounds are to be detected. The frame 42 has a generally flat bottom surface 48 to allow the matrix 41 to be placed on a flat surface, such as a bed, as explained below. The frame 42 is made from an acoustically damping material in order to dampen vibrations originating on the surface upon which the matrix 41 is placed.

The frame 42 is provided with one or more recesses 48. A microphone 42 is disposed in each of the recesses 50. A microphone 44, for example, the microphone 44a is suspended in its respective recess 48a by means of two or more elastic cords 52, so that the microphone 44a is not in direct contact with the frame 42. By not placing the microphones 42 in direct contact with the frame 42, any extraneous vibrations in the frame 42 are essentially not picked up by the microphones 2.

Each of the microphones 44 is provided with a spacer 43 made from an acoustically conducting material. The microphones 44 are disposed in the recesses 48 so that the spacer 43 of each microphone protrudes above the upper surface 46 of the frame 42, as shown in FIG. 6. As shown in FIG. 8, due to the elasticity of the cords 52, when a body surface 53 is applied to the upper surface 46 of the frame 42, each of the microphones 44 is applied to the body surface 53. In one embodiment of the invention, leads 56 extend from each microphone 44 to the exterior of the frame 42 via a system of channels 58 connecting each recess 48 with the exterior, so that the leads may be attached to signal processing circuitry (not shown), as required in any application. The microphone leads 56 extending from the frame 42 are collected into a single cable 68 that terminates in a plug 69 for connection to electronic circuitry for recording and/or analyzing voltage signals from the microphones 44. In another embodiment (not shown), the microphone leads are in the form of a printed circuit on the surface of the frame 42. In yet another embodiment, the microphones 44 are wireless, in which case each microphone includes a transmitter for transmitting signals to the electronic circuitry.

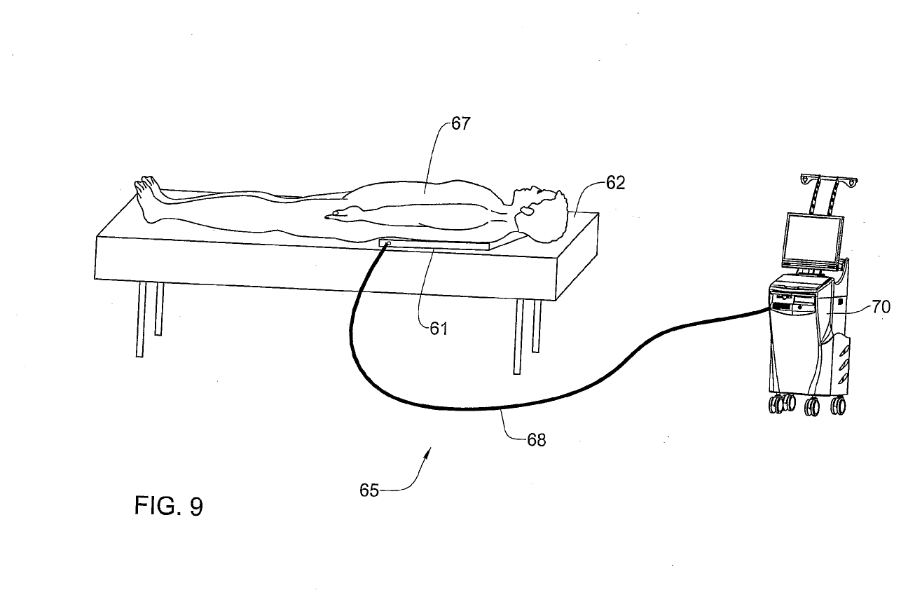

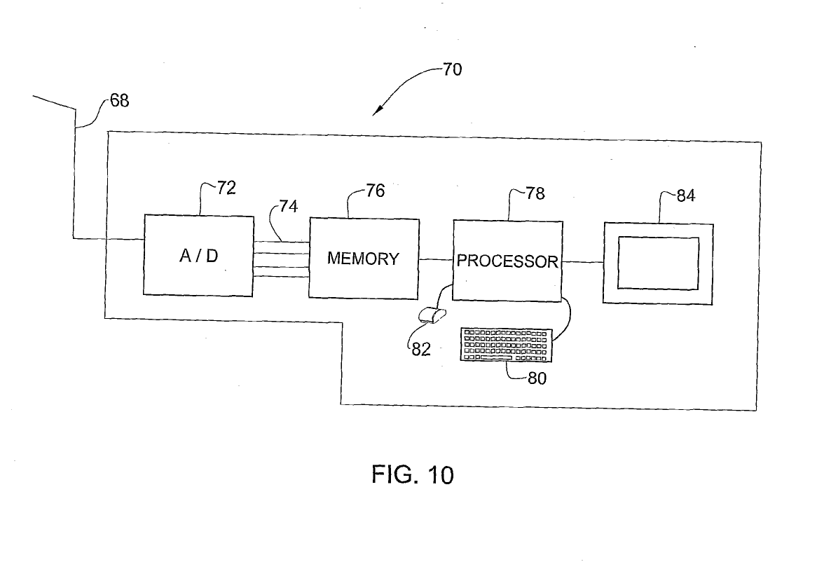

FIG. 9 shows a system 65 for recording and/or analyzing body sound signals, in accordance with the invention. The system 65 includes one or more microphone matrices 61 of the invention, for example the microphone matrix 30 described above in reference to FIG. 5 or the microphone matrix 44 described above in reference to FIGS. 6 and 7. Each microphone in the matrix 61 produces an analog voltage signal indicative of pressure waves arriving at that microphone. As shown in FIG. 10, in use, the matrix 61 is placed on a surface 62 such as an examination table or hospital bed. The upper surface of the microphone matrix 61, may be covered with an acoustically transparent disposable film (not shown) such as hospital grade “clean wrap” before a body surface, such as the back of an individual 64, is applied to the microphone matrix 61. The surface 62 may be completely flat as shown in FIG. 9, or may be articulated so as to allow the individual 64 to sit up during recording of body sounds.

As explained above, the microphones in the microphone matrix 61 are elastically biased to protrude above the upper surface of the matrix housing. Thus, with the back of the individual 64 properly positioned on the upper surface of the housing of the microphone matrix 61, at least some of the weight of the individual 64 is transferred to the upper surface of the support housing. In this way, the microphones are firmly applied to the individual's back without the need of an attachment system, such as straps, tape, or vacuum. Since the spacer of the microphone assembly is preferably made from an acoustically conducting material, respiratory tract sounds, or other body sounds originating in the thorax are detected by the microphones in the matrix 61. Extraneous vibrations originating outside the body such as vibrations due to movement of the individual or vibrations conducted through the surface 62, for example, due to the movement of other people or due to the operation of equipment in the vicinity of the surface 62, are essentially not detected by the microphones in the matrix since the microphone assemblies are connected to the housing or frame by a material that is not acoustically conducting.

The ability to apply the microphone matrix of the invention to a body region without the need for any fastening devices such as straps or vacuum, allows the microphone matrix to be applied to a body over a prolonged period of time. The system of the invention may thus be used for continuously monitoring and recording body sounds over a substantially unlimited period of time. In particular, the system may be used to continuously monitor body sounds such as reparatory tract sounds or cardiac sounds in an individual in an intensive care unit.

A cable 68 is connected to signal processing circuiting 70 for processing signals obtained by the microphones 44, as required in any application. The wires in the cable 68 provide each microphone 44 with a voltage that may be used for activating a preamplifier in the microphone, and also serve for transmitting a voltage signal to recording or analyzing circuitry when wires connected to a microphone are connected at another end to recording or analyzing circuitry, as described below. If the microphones 44 are wireless, the electronic circuitry 70 includes a receiver for receiving signals from the microphone transmitters.

FIG. 10 shows exemplary electronic circuitry 70 that may be used in the system 65 of the invention. Any method for analyzing body sounds may be used in the system of the invention. For example, the electronic circuitry for processing acoustic signals obtained from two or more microphones, as disclosed in U.S. Pat. No. 6,394,967 to Murphy, Kompis et al. supra, or U.S. Pat. No. 6,887,208 may be used in the system of the invention. In the circuitry shown in FIG. 10, the analog signals are digitized by a multi-channel analog to digital converter 72. The digital data signals 74, are input to a memory 76. Data input to the memory 76 are accessed by a processor 78 configured to process the data signals 74. The signals 74 may be denoised by filtering components having frequencies outside of the range of body sounds in the body region, for example, vibrations due to movement of the individual. Each signal 74 may also be individual to band pass filtering so that only frequency components in the signal within a range of interest are analyzed. An input device such as a computer keyboard 80 or mouse 82 is used to input relevant information relating to the examination such as personal details of the individual 64. A display screen 84 is used to display the signals 74 or the results of the processing.

1-19. (canceled)

20. A microphone matrix for recording body sounds, comprising:
(a) a flexible frame having a plurality of recesses or openings on a surface of the housing or frame; and
(b) one or more microphone assemblies, each microphone assembly comprising a microphone configured to record the body sounds, and each microphone assembly being disposed in a recess or opening in the frame, and wherein each microphone assembly is movable from an extended, spring biased position in which at least a portion of the microphone assembly protrudes beyond the surface of the frame to a retracted position in which the microphone assembly is deeper inside the recess.

21. The matrix according to claim 20 wherein the housing or frame is formed from an acoustically dampening material.

22. The matrix according to claim 20 wherein at least one microphone assembly is attached to the housing or frame by an acoustically dampening material.

23. The matrix according to claim 20 wherein at least one microphone assembly includes a spacer formed from an acoustically conducting material.

24. The matrix according to claim 20 further comprising one or more housings, each housing having a recess, wherein each microphone assembly is disposed in the recess of one of the housings and is supported by a tongue extending from a wall of the housing.

25. The matrix according to claim 20 wherein the frame has a plurality of apertures each aperture configured to receive a microphone assembly.

26. The matrix according to claim 20 wherein the body sounds are respiratory tract sounds.

27. The matrix according to claim 20 wherein the frame conforms to a body surface when the body surface is applied to the matrix.

28. The matrix according to claim 27 wherein the body surface includes at least a portion of a back.

29. The matrix according to claim 20 wherein channels are formed in the frame and microphone leads pass through the channels.

30. The matrix according to claim 20 wherein the microphones are wireless.

31. The matrix according to claim 20 wherein microphone leads are incorporated into a cable extending from the housing.

32. A method for recording body sounds comprising
(a) providing a microphone matrix for recording body sounds, the matrix comprising:
(i) a flexible frame having one or more recesses or openings on a surface of the housing or frame; and
(ii) one or more microphone assemblies, each microphone assembly comprising a microphone configured to record the body sounds, and each microphone assembly being disposed in a recess or opening in the frame, and wherein each microphone assembly is movable from an extended, spring biased position in which at least a portion of the microphone assembly protrudes beyond the surface of the frame to a retracted position in which the microphone assembly is deeper inside the recess.
(b) placing the microphone matrix on a surface; and
(c) applying a body surface of an individual to the matrix.

33. The method according to claim 32 wherein at least a portion of the weight of the individual is transferred to the matrix.

34. The method according to claim 32 wherein the matrix conforms to the body surface when the weight of the individual is transferred to the frame.

35. The method according to claim 32 wherein at least one microphone assembly moves from the extended spring biased position towards the retracted position when the weight of an individual is transferred to the microphone assembly.

36. The method according to claim 32 further comprising analyzing signals obtained by the one or more microphones.

37. The method according to claim 32, comprising continuously monitoring body sounds.

38. The method according to claim 32 wherein the body sounds are respiratory tract sounds.

39. A system for analyzing body sounds comprising:
(a) a microphone matrix for recording body sounds, the matrix comprising:
(i) a flexible frame having one or more recesses or openings on a surface of the housing or frame; and
(ii) one or more microphone assemblies, each microphone assembly comprising a microphone configured to record the body sounds, and each microphone assembly being disposed in a recess or opening in the frame, and wherein each microphone assembly is movable from an extended, spring biased position in which at least a portion of the microphone assembly protrudes beyond the surface of the frame to a retracted position in which the microphone assembly is deeper inside the recess; and
(b) electronic circuitry for analyzing signals obtained by the one or more microphones.

40. A microphone assembly for use in a microphone matrix according to claim 20.
